(19) United States
(12) Patent Application Publication
(10) Pub. No.: US 2024/0173373 A1
(43) Pub. Date: May 30, 2024

(54) NOVEL METHOD FOR THE PRODUCTION OF CONCENTRATED ALOE VERA COMPOSITIONS AND THERAPEUTIC USES FOR THE SAME

(71) Applicant: Desert Harvest, Inc., Elisworth, ME (US)

(72) Inventor: Heather Florio, Brooksville, ME (US)

(21) Appl. No.: 18/429,853

(22) Filed: Feb. 1, 2024

Related U.S. Application Data
(63) Continuation of application No. PCT/US2022/039055, filed on Aug. 1, 2022.
(60) Provisional application No. 63/233,130, filed on Aug. 13, 2021.

(51) Int. Cl. A61K 36/886

(52) U.S. Cl. CPC A61K 36/886; A61K 2236/13; A61K 2236/15; A61K 2236/31; A61K 2236/51; A61K 2236/53

(57) ABSTRACT

The invention includes a novel method, systems, and compositions for generating a concentrated Aloe vera extract. Embodiments include method, systems, and compositions for extracting a pharmaceutically active polysaccharidic substance from the Aloe plant. Additional embodiments may include therapeutic uses of the concentrated Aloe vera extract for the treatment of one or more diseases and conditions, including interstitial cystitis (IC).

### CROSS REFERENCE TO RELATED APPLICATIONS

The present application claims priority and is a continuation from PCT application serial no. PCT/US2022/039055, filed Aug. 1, 2022, which claims the benefit of and priority to U.S. Provisional Application No. 63/233,130, filed Aug. 13, 2021, the entire specification and figures of the above-referenced applications are herein incorporated by reference in their entireties.

### TECHNICAL FIELD

The inventive technology is directed to the field of extraction, concentration, and purification of various constituents components of plants. Specifically, the inventive technology includes novel extraction, concentration, and purification of various constituents components of the Aloe plant.

### BACKGROUND

Aloe vera belongs to the Asphodelaceae family of plants. It is a succulent, tender plant containing a high water content (˜99-99.5%). Solid content range from 0.5-1% and consists of a variety of active components such as fat and water soluble minerals, vitamins, simple/complex polysaccharides, organic acids, enzymes, and phenolic compounds. The Aloe leaf itself can be divided into two major parts, namely the outer green rind, including the vascular bundles, and the inner colorless parenchyma containing the Aloe gel. The three structural components of the Aloe vera pulp are the cell walls, the degenerated organelles and the viscous liquid contained within the cells. These three components of the inner leaf pulp have been shown to be distinctive from each other both in terms of morphology and sugar composition. It has been hypothesized that this heterogeneous composition of the Aloe vera pulp may contribute to the diverse pharmacological and therapeutic activities which have been observed for Aloe gel products.

Aloe vera contains two major liquid sources, a yellow latex (exudate) and the clear gel (mucilage). The dried exudate of Aloe barbadensis Miller leaves is referred to as Aloe. The commercial name is Curacao Aloe. It is composed mainly of aloin, Aloe-emodin and phenols. A number of phenolics, including anthraquinones and their glycosides, are known to be pharmaceutically active. The mucilaginous jelly from the parenchyma cells of the plant is referred to as Aloe vera gel. There are generally no anthraquinones to decompose and cause discoloration of the gel unless the gel is contaminated by an improper processing technique. Aloe vera gel is about 98.5% by weight water. More than 60% of the total solid is made up of polysaccharides of carbohydrate origin. Organic acids and inorganic compounds, especially calcium oxalate, account for the remainder of the solid.

Whole leaves, exudates and fresh gels of Aloe plants have been used for a variety of human afflictions. Evidence of their use as a medicinal remedy can be traced to the Egyptians of 400 BC. Aloe vera was also used to embalm the dead as well as to protect the embalmers from the death-causing agent. Other early civilizations used Aloe vera for skin care, relieving insect stings and bites, treating scratches, wound healing, hair loss, ulcerated skin and as a purgative. It was the traditional medicine of many cultures as an anthelmintic, cathartic, stomachic, and was used inter alia for leprosy, burns and allergic conditions.

Anti-inflammatory activity of Aloe vera gel has been widely reported by both oral testimonies and respected scientific journals. Also, noted in such testimonies and journals was the anti-inflammatory activity in polysaccharides extracted from fruit bodies or several fungi. The polysaccharides demonstrated significant inhibitory effect on carrageenan induced edema. One of the polymers, O-acetylated-D-mannan (T-2-HN), in addition demonstrated more marked inhibitory effect on scald hyperalgesia than phenylbutazone. The fact that the polysaccharide is said to be free from protein and lipids strongly suggests that the anti-inflammatory effect is due to the acetylated mannan only. Other researchers have also reported anti-inflammatory effects of complex polysaccharides, glycoproteins and sulfated polysaccharides.

### SUMMARY OF THE INVENTION

The present invention is directed to a process whereby the active chemical substance in the Aloe plant is physically extracted from whole Aloe leaves. The chemical substance is substantially non-degradable and can be administered in a therapeutically effective amount, that amount being the quantity required to produce a therapeutic effect such the reduction or prevention of symptoms of a disease or conditions such as interstitial cystitis (IC).

### BRIEF DESCRIPTION OF THE FIGURES

FIG. 1. Exemplary production flowchart for Aloe vera Gel 30× juice concentrate in one embodiment thereof;

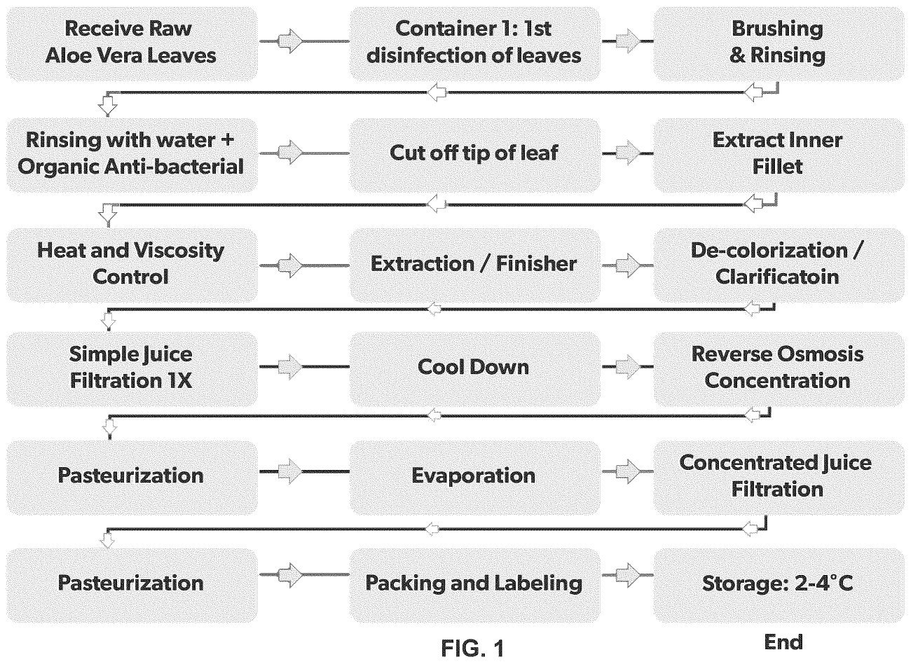

FIG. 2. Detailed stepwise production flowchart for Aloe vera Gel 30× juice concentrate in one embodiment thereof;

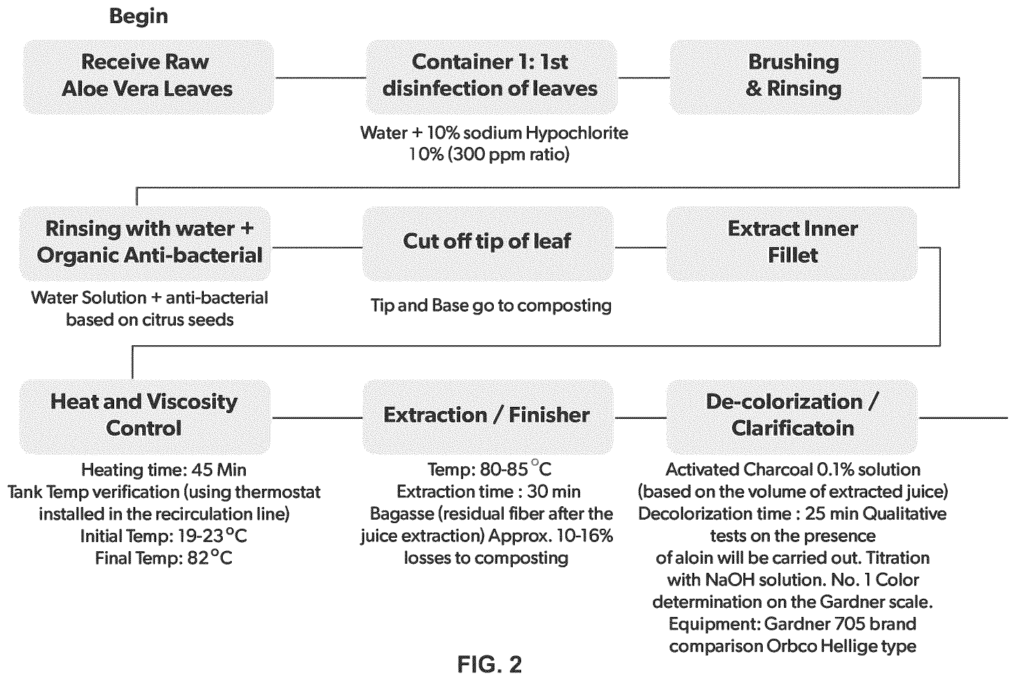

FIG. 3. Detailed stepwise production flowchart for Aloe vera Gel 30× juice concentrate in one embodiment thereof; and

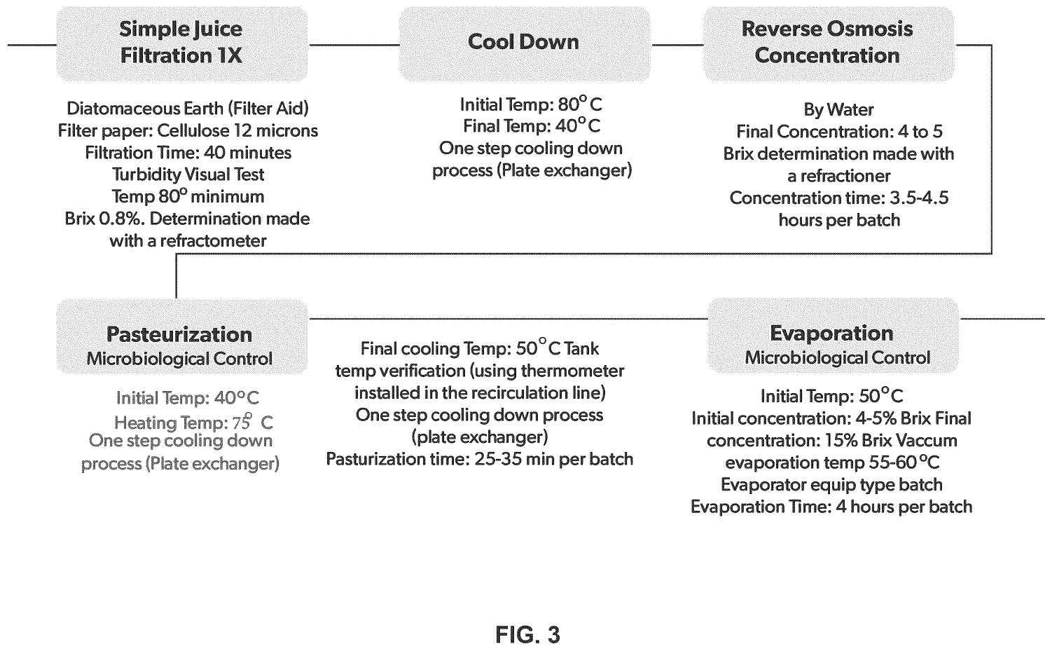

FIG. 4. Detailed stepwise production flowchart for Aloe vera Gel 30× juice concentrate in one embodiment thereof.

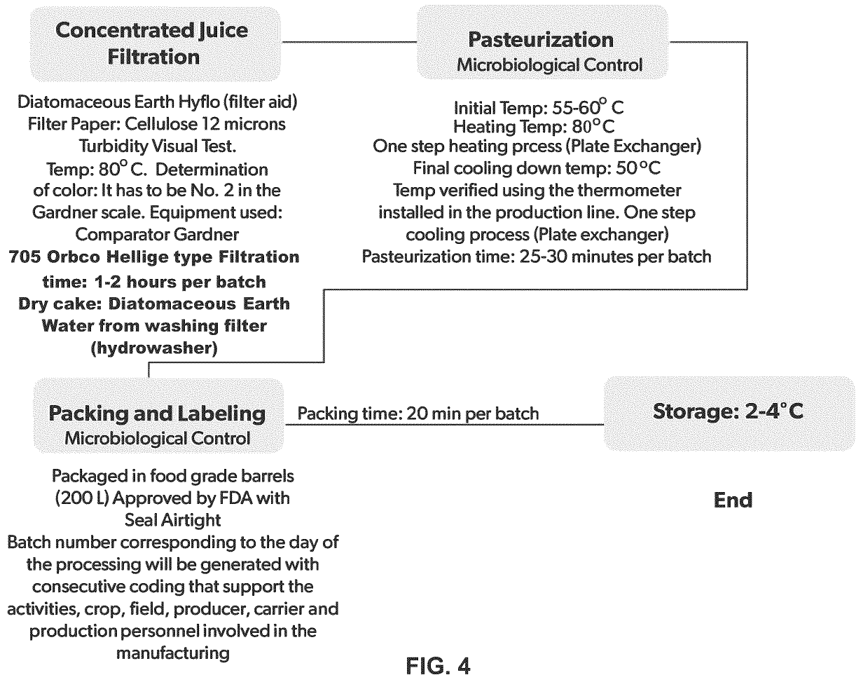

### DETAILED DESCRIPTION OF THE INVENTION

The present invention is directed to a process whereby the active chemical substance in the Aloe plant is physically extracted from whole Aloe leaves. The chemical substance is substantially non-degradable and can be administered in a therapeutically effective amount, that amount being the quantity required to produce a therapeutic effect such the reduction or prevention of symptoms of a disease or conditions, such as for interstitial cystitis (IC). The present invention is also directed to the active chemical substances in the Aloe plant in the form produced by the process described above. The active chemical substance has been found to be a substantially non-degradable lyophilized ordered linear polymer of acetylated mannose monomers. The mannose monomers are preferably bonded together by β-(1→4) bonds. The active chemical substance has been measured, standardized and characterized by analytical chemistry techniques.

The term active chemical substance as used herein means the substance, which is responsible for the wound healing and for the other beneficial properties of Aloe vera. In one preferred embodiment, active chemical substance(s) is used to treat interstitial cystitis (IC).

The process of the present invention is one for extracting the active chemical substance in the Aloe plant from a leaf of the Aloe plant, which process basically comprises at least the following steps: (a) washing an Aloe leaf in a bactericidal solution to remove substantially all surface dirt and bacteria and/or rinsing with an antibacterial compounds; (b) removing at least a first end portion from said washed leaf; (c) extracting the inner fillet from said cut and washed leaf; (d) removing rind from said leaf to produce a substantially anthraquinone-free Aloe gel fillet; (e) heating the Aloe gel fillet forming an aloe liquid or juice; (f) extracting and de-colorizing and clarifying the Aloe gel; (g) filtering the Aloe liquid and optionally cooling the Aloe liquid; and (h) concentrating the Aloe liquid; (i) pasteurizing the concentrated Aloe liquid; (j) evaporating the concentrated Aloe liquid; (k) filtering the concentrated Aloe liquid; and (l) optionally pasteurizing the concentrated Aloe liquid; and (m) optionally freeze-drying said concentrated Aloe vera liquid.

One skilled in the art will appreciate that one may obtain Aloe juice having solubilized matter from Aloe leaves in whatever manner possible, and then subject this juice to steps (c)-(k) to extract the active chemical substance. Indeed, one skilled in the art will appreciate that instead of steps (b), (c) and (d), one may instead (b) crush the washed Aloe leaves and (c) dialyze the crushed leaves chemically removing unwanted fractions, i.e., anthraquinones, minerals and acids, and the rind to produce a substantially anthraquinone-free gel that may then be subjected to steps (e)-(k) to extract the active chemical substance.

One skilled in the art will also appreciate that instead of steps (b), (c), and (d), one may instead crush the washed Aloe leaves and extrude anthraquinone-rich Aloe juice having solubilized matter and then subject the Aloe juice to steps (e)-(k) to extract the active chemical substance. The active chemical substance is effectively separated from anthraquinones and deleterious ions by this process since the anthraquinones and ions are water soluble and remain in the liquid solvent phase and do not precipitate.

One skilled in the art will also appreciate that instead of steps (b), (c), and (d), one may instead grind the whole washed Aloe leaves, filter out fibrous material, and homogenize the remainder to produce anthraquinone-rich Aloe juice having solubilized matter. The Aloe juice can then be subjected to steps (e)-(k) to extract the active chemical substance. The active chemical substance is effectively separated from anthraquinones and deleterious ions by this process for the reasons noted above. One skilled in the art will appreciate that an additional process for extracting the active chemical substance in the Aloe plant from a leaf of the Aloe plant without the use of a lower aliphatic polar solvent, such as ethanol, methanol or propanol.

With respect to step (a), the removal of “substantially all surface dirt and bacteria” means (1) removal of dirt to the extent that remaining dirt is less than 0.1% by weight of the weight of the leaf and (2) killing such surface bacteria that the remaining surface bacteria are less than 100 count per gram of leaves. Furthermore, the preferred process may further comprise the step of ultrafiltration in order to osmotically adjust the Aloe juice or Aloe vera fraction, or to reduce even further the levels of anthraquinones to less than 5 parts per million (ppm), even down to less than 100 parts per billion (ppb) by weight. These steps enable the processor to use large or small leaves (even less than one year old) because the polymer size found in the mature leaves can be selected and processed out of smaller, immature leaves.

With respect to step (a), the removal of “substantially all surface dirt and bacteria” may include manually brushing, washing, and/or rinsing dirt and bacterial components from the Aloe vera leaves. In another embodiment, step (a) may include introducing the raw Aloe vera leaves to an anti-microbial solution, preferrable a sodium hypochlorite solution. In this embodiment, the anti-microbial solution may include a solution of 10% sodium hypochlorite in water with an approximately 300 ppm ratio. In alternative embodiment, the anti-microbial solution may include an organic anti-microbial solution, such as an acid derived from citrus fruits and other natural comparable anti-microbial compounds.

As an additional preferred embodiment, the washing step of the process may comprise washing the substantially anthraquinone-free Aloe gel fillet in a tumbler washer prior to grinding said fillet. More preferably, in all of the above processes, Aloe leaves or whole plants may be collected from the field sufficiently clean to eliminate a washing step. The dried, precipitated active ingredient, optionally, may be irradiated by gamma or microwave radiation whereby said active chemical substance is sterilized and preserved. Accordingly, the present invention is believed to provide new and improved methods for the production of Aloe vera products.

With respect to step (e), in a preferred embodiment, the substantially anthraquinone-free Aloe gel fillet may be heated for approximately 45 minutes with an initial temperature between 19-23° C. and a final temperature of approximately 82° C. This heating steps may be monitored using a thermostat installed in a recirculating line.

With respect to step (f), in a preferred embodiment, the Aloe vera liquid may be extracted with a portion of the insoluble portion of the plant, such as the residual fibers and other components, left behind as bagasse. In a preferred embodiment, between 10-16% of the Aloe vera gel may be extracted as bagasse; and further, optionally composted or otherwise finally disposed. Step (c) may be accomplished at a temperature between 80-85º C. for approximately 30 minutes.

With respect to step (f), the Aloe vera liquid may undergo de-colorization and clarification steps. In this embodiment, the allow Aloe vera liquid may be introduced to a solution of activated charcoal, which may preferably include a 0.1% solution activated charcoal based on the volume of extracted juice. The Aloe vera liquid and solution of activated charcoal may remain in contact for approximately twenty-five (25) minutes. During, or after step (f), qualitative tests may be performed on the Aloe vera liquid to determine the presence and quantities of the components aloin. Further, Aloe vera liquid may be titrated with a base solution, such as a solution of NaOH. During, or after this process, the clarified Aloe vera liquid may be subjected to a color determination based on the Gardner scale.

One of the advantages of the instant process is that damaged leaves previously considered unusable due to strong winds or poor collection techniques can be processed, and the undesirable contaminants can be filtered out. With respect to step (g), the filtration step may incorporate membrane technology that allows the selection of filters with different pore sizes, depending on the condition of the cut Aloe leaves, that can accomplish any combination of the following: (1) A small pore size filter (preferably 100 Daltons) that separates out water and salts from the Aloe vera gel, if needed. (2) Larger pore size filters (preferably 500 Daltons) that can separate out the acids from the Aloe vera gel, if needed. (3) Even larger pore size filters (preferably 2000 Daltons) that can separate the yellow sap components from the Aloe vera gel, if needed. (4) And even larger pore size filters (preferably from 10,000-100,000 Daltons) that can size the gel matrix polymers, and divide them out by molecular weight.

A Romicon® 4-column (Romicon Co., 100 Cummings Park, Woburn, Mass. 01801, Model No. HF4 SSS, Membrane Type PM50, Membrane Nos. H526.5-43-pm50) as an ultrafiltration device is recommended.

In one preferred embodiment, filtration of the clarified Aloe liquid may be through a cellulose filter having a 12-micron pore size over approximately forty (40) minutes. In an optional set of steps, the tributary of the Aloe liquid may be evaluated. In these optional steps, the liquid is subjected to a refractometer to evaluate the sugar content based on a Brix scale. In a preferred embodiment, the liquid will be less than one degree, or optionally 0.8% Brix.

With respect to step (j), the evaporation step may be accomplished at an initial temperature of 50° C., having an initial concentration of sugars between 4-5% Brix, and a final concentration of sugars at 15%. The evaporation step may occur through vacuum evaporation at a temperature between 55-60° C., and may further preferably be accomplished in a batch system.

With respect to step (k), the filtering step may be as described above using a cellulose filter having a 12-micron pore size over 1-2 hours in preferably a batch process. In a preferred embodiment, the Aloe vera liquid may be No. 2 in the Gardner scale with respect to color and clarity of the liquid.

The present invention also is believed to provide new and improved processes for preparing extracts of the leaf of the Aloe vera plant, which maximize the concentration of certain components characteristic of particular portions or segments of the leaf while minimizing or eliminating certain components characteristic of other portions or segments of the leaf. The present invention also provides new and improved processes for preparing extracts from the leaf of the Aloe vera plant, which are low in concentration of yellow sap of the leaf.

The present invention finally is thought to provide a method for extracting the active chemical substance in Aloe vera gel. This chemical substance has utility as a non-toxic immune stimulating compound. The substance shall hereinafter be referred to as the concentrated extract may contain a quantity of mucopolysaccharide, such as acemannan. As mentioned above, mucopolysaccharides have been found to be a substantially nondegradable lyophilized linear polymer of acetylated mannose monomers which standardized and characterized by analytical chemistry techniques.

### EXAMPLE 1: TREATMENT OF IC USING CONCENTRATED ALOE VERA LIQUID

IC is a heterogeneous chronic disease characterized by suprapubic pain, perceived to derive from the urinary bladder, and other symptoms including urinary frequency, urgency, and nocturia. Most commonly affecting women, this disease significantly reduces the quality of life for many people and increases the symptom burden with respect to many activities of daily living. In addition, accurately diagnosing and treating IC has been found to be challenging for both clinicians and patients alike due to numerous theories regarding etiology and pathophysiology, as well as its similar presentation to other urologic conditions. Therefore, many patients often resort to alternative therapies for relief of their IC-associated symptoms. One prevalent hypothesis regarding etiology for IC is the thinning of the bladder epithelium due to breakdown of the glycosaminoglycans (GAG) layer, which is substantially responsible for bladder impermeability to various toxins and inflammatory responses.

One of the active chemical substances of the invention includes glycosaminoglycans, which comprise a class of mucopolysaccharides and which are known to have hydro-repellant properties. Within the bladder, alterations in the GAG layer can expose the urothelium to many toxic urinary agents, which, in turn, can penetrate into the bladder wall. When this occurs, it is hypothesized that a sequential immune response is initiated in the submucosa in which nerve terminals produce inflammatory mediators causing mast cell degranulation and histamine secretion with resulting vasodilation and inflammatory exudate. Consequently, this inflammatory response can generate severe bladder pain, which acts as the distinguishing symptom for patients diagnosed with interstitial cystitis. In one embodiment, natural occurring mucopolysaccharide containing glycosaminoglycans, and other constituents, may be extracted from the Aloe vera plant according to the process of the invention and used to treat IC by replenishing the damaged GAG layer at the mucosal surface of the bladder and preventing irritants from acting upon it.

In a preferred embodiment, concentrated Aloe vera generated from the process described herein may be paced in a capsule contains 600 mg pure, freeze-dried Aloe vera with a minimum of 200 mg of glycosaminoglycans per capsule. The Aloe vera appears as a crystalline powder in appearance, ranging in color from cream to light beige. The moisture content is 8%, maximum pH (1% solution at 25° C.) is 4.5-4.7. Each capsule contains an additional 20 mg of calcium carbonate to raise the pH of the Aloe powder.

Following the prescribed regime, eligible participants may be instructed to self-administer

their randomly assigned active or placebo medication orally at a dose of three (3) capsules BID (morning and evening, 10 to 12 hours apart) for the first month, three (3) capsules TID (morning, afternoon, and evening, 6 to 8 hours apart) for the second month, four (4) capsules TID (morning, afternoon, and evening, 6 to 8 hours apart) for the third month, and reduce the dosage by two (2) capsules per week for the remaining four (4) weeks of the trial as follows:

First week=10 capsules per day (4 in the morning, 2 in the afternoon, 4 in the evening)

Second week=8 capsules per day (4 in the morning, 4 in the evening)

Third week=6 capsules per day (3 in the morning, 3 in the evening)

Fourth week=4 capsules per day (2 in the morning, 2 in the evening)

| | Study | | | | |
|---|---|---|---|---|---|
| | Drug | Month 1 | Month 2 | Month 3 | Month 4 |
| Study | Aloe | 3 capsules | 3 capsules | 4 capsules | Reduce dosage by |
| Group | Vera | BID | TID | TID | 2 for 4 weeks |
| Control | Placebo | 3 capsules | 3 capsules | 4 capsules | Reduce dosage by |
| Group | | BID | TID | TID | 2 for 4 weeks |

All doses may be ingested with eight (8) ounces of water per dose, but can be taken with or without food. Participants should administer their evening dose at least two (2) hours before their bedtime.

The above study may be designed as a randomized, double-blind, placebo-controlled clinical trial with a two-arm parallel group arrangement to determine the safety and efficacy of super-concentrated, freeze-dried Aloe vera in minimizing the symptoms associated with interstitial cystitis. Approximately 100 participants may be recruited over a period of 24 months. Each eligible participant may be randomized into study and control arms in a 4:1 ratio, favoring the active treatment, in which they will self-administer their assigned capsules orally over a sixteen-week period. The dosage can be increased for the first three (3) months of the study followed by a decrease in the dosing regimen during the fourth month.

In this embodiment, concentrated Aloe vera, preferably in capsule form, may serve to decrease the symptoms associated with interstitial cystitis including suprapubic pain, urinary frequency, nocturia, dysuria, and urinary urgency. In this embodiment, concentrated Aloe vera, preferably in capsule form, may alter gene expression in whole blood samples from participants that may harbor any biological markers (i.e., expressed genes) common across many patients with

IC, allowing an evaluation of the blood sample of a patient presenting to a clinician with IC-like symptoms and circumvent the need for invasive diagnostic therapies or procedures.

All eligible and consenting participants may attend six (6) visits over a sixteen (16)-week period. Participants may be screened for eligibility during Visit 1 of the study and randomization will occur during Visit 2. Treatment numbers may be assigned to participants sequentially and the study medication may be administered in a double-blind fashion with identical capsules and packaging. Four 24-hour voiding diaries will be administered, and the participants may be instructed to complete one diary every week for four (4) weeks until they return for Visit 3. The dosage may continue to be increased by three (3) capsules every month until Visit 5 in which participants may be instructed to decrease their dose by two (2) capsules every week for four (4) weeks. Similar procedures will occur for Visit 6 compared to Visit 1; and then, the study may conclude for the participant. During Visit 6, the participant may be made aware of which group they were randomly assigned.

A full description of the Visit Calendar that may be utilized throughout the study is shown below:

| Visit | Location | Description |
|---|---|---|
| Visit 1 - | Clinic | Informed consent obtained. Physical exam performed and laboratory assessments |
| Screening | | collected. Patch skin test performed to confirm eligibility. Questionnaires completed |
| | | and Visit 2 scheduled within 7-10 days. |
| Visit 2 | Virtual | Participant is randomized to receive either study or control capsules and instructed to |
| in 7 to 10 days | | self-administer three (3) capsules BID for four (4) weeks. Four (4) electronic 24- |
| | | Hour Voiding Diaries are dispensed, one diary to be completed once a week for four |
| | | (4) weeks. Participant is administered questionnaires to complete before their next |
| | | visit. Visit 3 scheduled within four (4) weeks ± 3 days. |
| Visit 3 | Virtual | Review 24-hour voiding diaries. Participant is instructed to increase dose by self- |
| in 4 weeks ± 3 | | administering three (3) capsules TID for four (4) weeks. Questionnaires and one |
| days | | electronic 24-Hour Voiding Diary is dispensed, to be completed before the next visit. |
| | | Visit 4 scheduled within four (4) weeks ± 3 days. |
| Visit 4 | Virtual | Review 24-hour voiding diary. Participant is instructed to increase dose by self- |
| in 4 weeks ± 3 | | administering four (4) capsules TID for four (4) weeks. Questionnaires and one |
| days | | electronic 24-Hour Voiding Diary is dispensed, to be completed the day before the |
| | | next visit. Visit 5 scheduled within four (4) weeks ± 3 days. |
| Visit 5 | Virtual | Review 24-hour voiding diary. Participant is instructed to decrease dose by two |
| in 4 weeks ± 3 | | (2) capsules every week for four (4) weeks. Questionnaires and one electronic 24- |
| days | | Hour Voiding Diary is dispensed, to be completed before the next visit. |
| | | Visit 6 scheduled within four (4) weeks ± 3 days. |
| Visit 6 - or ET | Clinic | Review 24-hour voiding diary. Physical exam performed and laboratory assessments |
| in 4 weeks ± 3 | | collected. Questionnaires are administered, to be completed in clinic. End of clinical |
| days | | trial. |

What is claimed is:

1. A method of producing a concentrated Aloe vera extract comprising:
(a) washing an Aloe leaf in a bactericidal solution to remove substantially all surface dirt and bacteria and/or rinsing with an antibacterial compounds;
(b) removing at least a first end portion from said washed leaf;
(c) extracting the inner fillet from said cut and washed leaf,;
(d) removing rind from said leaf to produce a substantially anthraquinone-free Aloe gel fillet;
(e) heating the Aloe gel fillet forming an Aloe vera liquid;
(f) extracting and de-colorizing and clarifying the Aloe vera liquid;
(g) filtering the clarified Aloe vera liquid and optionally cooling the Aloe vera liquid; and
(h) concentrating the Aloe vera liquid;
(i) pasteurizing the concentrated Aloe vera liquid;
(j) evaporating the concentrated Aloe vera liquid;
(k) filtering the concentrated Aloe vera liquid; and
(l) optionally pasteurizing the concentrated Aloe vera liquid; and
(m) optionally freeze-drying said concentrated Aloe vera liquid.

2. A composition comprising an Aloe vera concentrate produced by the process of claim 1.

3. A method of treating interstitial cystitis (IC) the method comprising administering a therapeutically effective amount of the Aloe vera concentrate of claim 2 to a subject in need thereof.

4. A composition for treating interstitial cystitis (IC) comprising a therapeutically effective amount of the Aloe vera concentrate of claim 2.
